(19) United States
(12) Patent Application Publication
(10) Pub. No.: US 2008/0319315 A1
(43) Pub. Date: Dec. 25, 2008

(54) Method of reducing interferences in positron emission tomography

(75) Inventors: Gerald Kolodny, Newton, MA (US); Gethin Williams, Newton, MA (US)

Correspondence Address: Edwards Angell Palmer & Dodge LLP, P.O. Box 55874, Boston, MA, 02205, US

(73) Assignee: Beth Israel Deaconess Medical Center, Boston, MA (US)

(21) Appl. No.: 12/156,724

(22) Filed: Jun. 4, 2008

Related U.S. Application Data
(60) Provisional application No. 60/933,609, filed on Jun. 7, 2007.

(51) Int. Cl. A61B 6/00; A61K 49/00

(52) U.S. Cl. 600/431; 424/9.1

(57) ABSTRACT

Methods and compositions are disclosed that lower the uptake of the radiopharmaceutical FDG, a glucose analog, by brown adipose tissue and the myocardium in FDG-PET/CT scans. The composition is substantially carbohydrate free and includes high levels of lipids. The method uses the composition and doses at particular times before the scan. The method has an advantage over fasting methods since it reduces the uptake by brown adipose tissue and/or myocardium that can be mistaken for cancer on FDG-PET or PET//CT scans or obscures sites of cancer adjacent to the heart. In addition, this permits the identification, monitoring and treatment of coronary artery disease on PET or PET/CT scans.

### RELATED PATENT APPLICATIONS

This is a utility patent application which claims the benefit of U.S. provisional patent application No. 60/933,609, filed on Jun. 7, 2007, the entirety of which is herein incorporated by reference.

### BACKGROUND

The present invention relates to improvements in Positron Emission Tomography (PET). More particular, the present invention concerns compositions and methods for use in improving discrimination of cancerous or other tissues of interest in FDG-PET scans, in identification of inflamed coronary plaque and in monitoring treatment for coronary artery disease.

Cancer is a major health problem in the United States. FDG-PET/CT has emerged as an important imaging modality to diagnose, stage, and monitor treatment of a wide variety of cancers. Patients are injected with 18F-2-fluoro-2-deoxy-D-glucose (FDG), a radioactively labeled glucose analog, that is preferentially taken up by cancer cells due to the Warburg effect. Glucose uptake and glycolysis are greatly increased in tumors. The amount of FDG uptake and anatomic location is useful in diagnosis and treatment monitoring since it can define the aggressiveness of the cancer and its stage. By serially scanning patients after treatment is initiated, sites of FDG-uptake, their size and the intensity of uptake can be monitored, during and after treatment. The computed tomography (CT) scan is used as part of the procedure to localize and identify sites within the body with high FDG uptake. While the PET scan provides excellent information on uptake, it does not provide good information on anatomic location. The CT scan provides the physical location information but not the uptake information. Using the combined scans leads to both image data sets and markedly enhanced predictive information.

There is also some indication that FDG-PET scans can be used to determine the presence and extent of coronary artery disease (CAD) as well. The inflamed cells in CAD, particularly the atherosclerotic cells, have a higher uptake of glucose than normal adjacent cells. Since these cells will take up FDG as if it were glucose, the radioactive FDG will be concentrated in the cells having the highest glucose uptake.

Problems arise when FDG-uptake can be exhibited by normal structures and normal physiology, causing false interpretations. A false interpretation may lead to the patient receiving unneeded treatment including chemotherapy, irradiation, and biopsies. Further, normal uptake can obscure disease leading to false negative reports and patients not receiving needed treatments. Two normal sites which show high uptake of FDG are the brown adipose tissue and the myocardium. These both have high uptake of FDG, leading to possible false positive interpretations of the PET/CT scans and obscuring of tumors. It is also possible that by decreasing confounding mediastinal, cervical and paraspinal FDG-uptake, sites of inflammation including coronary disease can be visualized.

Current methods for patient preparation of FDG-PET/CT scans include a fast of 4-12 hours prior to dosing with FDG. Consequences of this method include variable to high myocardial FDG-uptake that can cause both false positives (the nodular appearance mimicking cancer) and false negatives, as true sites of cancer are obscured. Brown adipose tissue (or brown fat), which can occur in up to 30-40% of PET scans, can, like the myocardial uptake, cause false positive and false negative interpretations of the PET scans. Brown fat generally has a nodular appearance that can both mimic cancer and obscure sites of cancer, greatly limiting and compromising scans.

Methods to decrease myocardial uptake of FDG in FDG-PET/CT have included further fasting and beta-blockers. In addition, there has been some mention of a high fat solid diet with protein and low carbohydrate to reduce interferences but no timelines or explicit composition of the high fat/low carbohydrate has been defined.

Methods to decrease uptake of FDG by brown adipose tissue have included benzodiazepines, propanolol, reserpine and warming, all with variable results. Apart from warming, all of these have the disadvantages of pharmacological side effects, especially in the pediatric population, which is a population which shows the highest incidence of brown adipose tissue problems on FDG-PET/CT.

Since uptake of FDG by brown adipose tissue and the myocardium reduces the pool of FDG and thus lowers the sensitivity of FDG-PET to detect lesions of interest, methods that would decrease brown adipose tissue and myocardium FDG-uptake should increase the sensitivity of PET scans. A non-drug method that effectively reduced myocardial and/or brown adipose tissue FDG-uptake would offer the potential to decrease false positive and false negative studies, improving overall sensitivity.

### SUMMARY OF THE INVENTION

The present invention utilizes the finding that ingestion of a high fat/no carbohydrate/low protein, aqueous emulsion several hours prior to administration of FDG and conducting the PET scanning improves the sensitivity of the scan. In particular, having a patient take a high fat/no carbohydrate solution, rather than the usual fast prior to scanning, reduces uptake of FDG by the myocardium and brown adipose tissue. This finding is not only useful in scans for tumors but it allows inflammation in the coronary arteries to be detectable by FDG-PET/CT scans, thus allowing detection of coronary artery disease, monitoring drug treatment of coronary artery disease, and detecting and monitoring in-stent retenosis.

The methods and compositions of the invention all utilize a composition of lipids, preferably in water, that is ingested about 1-10 hours, preferably 2-7 hours and more preferably 3-5 hours before the FDG is given. The patient should not eat for at least 6 hours before the test, before taking the composition. While the composition is generally given orally, in certain circumstances it may be given by gavage or other form of enteral feeding. It may be semi-solid or in a capsule but normally it is in the form of a beverage. The composition should be substantially free of carbohydrates.

The composition should contain 5-75% by volume lipids, preferably 20-60% or 30-50% lipid and 25-95% by volume of a carrier, preferably water. Small additional amounts of other materials, such as flavorings and emulsifiers may be added. While a small amount of protein may be added, it is not necessary and in some circumstances may be detrimental. The preferred lipids are vegetable or fish oil lipids rather than animal lipids. This leads to a higher proportion of highly unsaturated n-6 and n-3 lipids and fewer of the short chain (C₁₀ or fewer carbons) or saturated fats. The lipids may be in the form of free fatty acids, mono-, di- and triglycerides, structured lipids having the combination of lipids on the glycerol backbone engineered for best effect, or a combination of any of the above.

Preferred compositions are taken optimally between 3 to 5 hours before FDG radiopharmaceutical injection. If shorter chain fatty acids are used, the time may be shortened. The composition should contain about 5-95 g free fatty acids (which are preferably long-chain (C₁₄ or higher) fatty acids) and 5-95 g mono-, di- and triglycerides (preferably of long-chain fatty acids) for a total of 10-150 g of lipid constituent. The lipids can be mixed with water or other non-carbohydrate containing liquid to provide a beverage or semi-solid. Compositions having 20-50% lipid are preferred. By including a mixture of free fatty acids and mono-, di- and triglycerides, brown fat is provided thermogenic precursors that last after the post-prandial insulin peak, thus minimizing the need of the brown fat to metabolize glucose and FDG.

Lipids preferred for use in the composition are C₁₆ or greater monounsaturated fatty acids such as palmitoleic, oleic, elaidic, and ricinoleic and polyunsaturated fatty acids such as linoleic, gamma-linolenic, arachidonic, eicosapentaenoic, and docosapentaenoic. In addition, medium chain triglycerides (C₈-C₁₂) are useful in the form of either free fatty acids or as part of a triglyceride or structured lipid. Sources of these fatty acids and mono-, di- and triglycerides can be vegetable oils such as canola or flaxseed oil or fish oils such as menhaden oil. Because many of these oils have strong odors or strong tastes, flavorings, primarily oil based flavorings, can be added for palatability.

This invention also pertains to a method for providing an individual who is undergoing a FDG-PET procedure (with or without CT) with a dietary supplementation that (1) decreases FDG-uptake in brown adipose tissue, (2) decreases FDG-uptake in myocardium and (3) increases FDG concentration in a patient PET or PET/CT scan. This permits increased sensitivity of the scan, a decrease in false positive and false negative scans and the ability to identify inflamed coronary artery plaques. The method can be carried out by administering to the individual the composition described herein, in effective doses and in the timeline described.

### BRIEF DESCRIPTION OF THE DRAWING

FIG. 1 is a photograph showing FDG-PET/CT scans of the same patient after fasting (FIG. 1A) versus following the protocol of the invention (FIG. 1B).

FIG. 2 is a graph showing the effect of one protocol of the invention on FDG-uptake by the myocardium, plotting the maximum Standard Uptake Values (SUV) for the composition given at various times before the injection of the radiopharmaceutical.

FIG. 3 is a spreadsheet showing the maximum myocardial uptake of FDG after ingestion of three formulations compared with fasting after a low carbohydrate meal.

### DETAILED DESCRIPTION OF THE INVENTION

The present invention relates to a method and composition formulated to supply a person metabolic substrates at a specific time prior to an FDG-PET study that will minimize FDG-uptake in sites that may confound diagnostic accuracy. Reducing interfering sources of FDG-uptake in scans can improve diagnostic accuracy by minimizing false positives (mistaking physiological FDG-uptake for cancer FDG-uptake) or false negatives (physiological uptake may obscure sites of disease). Two organs in the body that have been established to cause such problems in FDG-PET scan using current patient preparation protocols are brown adipose tissue and the myocardium. Since brown adipose tissue does not occur on every scan, it is a particular problem because it is not clear if the high uptake region is a true positive or a false positive. In addition, if brown adipose tissue is present, high uptake of FDG can reduce the uptake of the cancerous or inflammatory cells that are being sought or it can physically block an area from being visualized. Although the myocardium is not seen in every scan, the method generally used to assess how it will affect an FDG-scan includes a measurement of the Standardize Uptake Value (SUV). High values indicate obscuration of cancer and low values indicate a low background. By reducing the uptake of the brown adipose tissue and myocardium in all scans using the methods and composition of the present invention, improved discrimination is possible.

Other methods that have been tried to decrease brown adipose tissue FDG-uptake for PET studies have not been successful. For example beta-blockers, diazepam and fentanyl have been used to decrease brown adipose tissue FDG-uptake but with variable results. Further these pharmaceuticals may have side-effects and may be problematic in pediatric populations where brown adipose tissue is frequently seen in FDG-PET studies.

The prior methods of preparing the patient for a FDG-PET scan involved fasting for about 8 hours prior to the scan. Because glucose and other carbohydrates can be taken up in lieu of the FDG, the theory was that all of the glucose would have been metabolized and thus the cancer cells would take up the FDG faster than the other cells. However, as noted, certain cells, particularly those in the brain, myocardium and brown adipose tissue, also take up FDG more rapidly than other cells, leading to false positive and negative studies. Another variant that has been tried was to have the patient eat an “Atkins-like” meal about 6-8 hours before the scan. This meal would have heavy protein (and the attendant high fat) but low carbohydrates. While this may provide some improvement, it does not solve the problems of the myocardium or brown adipose tissue uptake.

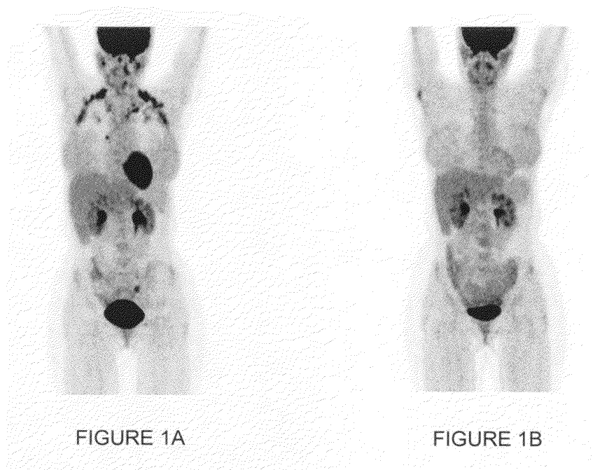

FIG. 1 is a photograph showing the effects of the method and composition of the invention versus the traditional fasting. FIG. 1A shows a FDG-PET/CT scan of a patient after fasting. Brown adipose tissue nodules (10) are evident near the neck region because of the high uptake of FDG. In addition, the heart (20) is also evident because of its high FDG uptake. The high uptake by the brown adipose tissue nodules and the heart can block visualization of cancer cells or inflammation of the arteries that could be a sign of cardiovascular disease. In contrast, FIG. 1B shows a similar scan of the same patient using a formulation of the present invention. The patient had a high fat, low carbohydrate meal in the evening and a drink consisting of 50 mL canola oil, 0.5 mL peppermint oil as a flavoring, and water to bring the volume to 200 mL about 5 hours before the scan. As is evident, the brown adipose tissue and heart are barely visible. Thus, the problem with confounding FDG uptake and visualization is minimized.

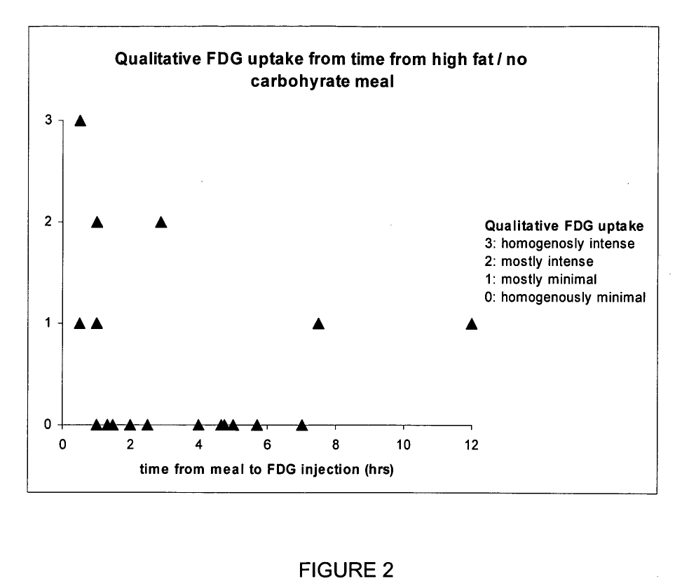

FIG. 2 is a timing graph, showing FDG-uptake by the myocardium, shown as Standard Uptake Values (SUV) versus time between the ingestion of the drink and the injection of the FDG. Although differences in the fatty-acid chain length can influence timing, the optimal timing of ingestion is 3-5 hours before the administration of the FDG radiopharmaceutical, although 1-10 hours can be used. The decrease of SUV for brown adipose tissue would be expected to follow the same time course as is shown in FIG. 2 for the myocardium. This decrease results from the natural physiological stimulation of insulin secretion by food intake, which rises to a peak and then usually decreases to baselines levels by three hours after a meal. Since insulin stimulates the GLUT-4 receptors in muscle and fat, high FDG uptake will be seen in the heart and brown adipose tissue if FDG is injected during a time when insulin is elevated. This is not the desired or optimal result. By administering the aforementioned lipid composition 3-5 hours prior to injection of FDG, the post-prandial insulin peak subsides to near baseline and FDG-uptake in the brown adipose tissue and myocardium is minimized. Tumor cells generally have glucose/FDG uptake that is unaffected by fatty acid levels, so that FDG is rapidly taken up by the cancer cells while the surrounding normal cells, including the brown adipose tissue and myocardium, have decreased uptake, leading to better images of cancer sites against a reduced background. Similarly, coronary artery disease (CAD) is more evident because the inflammatory cells associated with CAD still obtain high levels of FDG while the interference from the heart muscle and brown adipose tissue is reduced. There is also some indication that fat in general, including brown adipose fat, may have an effect on glucose metabolism and therefore may be related to obesity and diabetes.

TABLE 1

| | FASTING | HIGH FAT |
|---|---|---|
| Mean | 6.3 | 2.3 percent |
| Variance | 3.7 | 1.1 |
| Observations | 12.0 | 6.0 months |
| Pooled Variance | 2.8 | |
| P(T <= t) one-tail | 0.00014 | |

Table 1 shows the frequency of brown adipose tissue noted in scans of patients prepared (1) by fasting and (2) by a composition within the scope of the invention. These are FDG-PET/CT scans (n=1229; 52% male; age 58+−16y; blood glucose level 109+−32). Brown adipose tissue was evident on 6.3% of scans. FDG-PET scans using a high fat method (n=741; 53% male; age 58+−16y; blood glucose level 99+−43) demonstrated brown adipose tissue with an uncorrected frequency of 2.8% (p<0.0002). Only the blood glucose level was significantly different between groups (p<<0.0001). As is evident, the number of patients showing uptake of FDG by brown adipose tissue decrease dramatically using the present composition, leading to fewer false positive results. In addition, although not evident from the Table, the amount of uptake in the locations of brown adipose tissue decreased as well, causing less obscuring of possible tumors and thus fewer false negative studies.

A variety of lipids can be used in the composition of the invention. These include free fatty acids, mono-, di- and triglycerides and structured lipids. In addition, the compositions can contain aqueous or oil flavorings and emulsifying agents. It also appears that proteins may be included, up to about equal the amount of the lipids in the composition. What is clear, however, is that the composition should be substantially free of carbohydrates, particularly sugars. However, complex carbohydrates that are not broken down to glucose or other sugars during the time frame from ingestion to taking the scan may be included.

Free fatty acids can be obtained from a variety of vegetable and animal sources consistent with the Food and Drug Administration (FDA) requirements for food-grade products. Fatty acids can be classified as short (C₈ or less), medium (C₁₀-C₁₂) and long chain (Cl₄ or greater). The fatty acids may be further divided into classes that include polysaturated, monounsaturated and saturated depending on the number of double bonds, or omega-3 (also called n-3), omega-6 (n-6), and omega-9 (n-9) depending on the location of the double bonds. The n-6 fatty acids are found from many sources, while the n-3 fatty acids are primarily from fish oil, fungal oils or certain vegetable oils such as flax or rapeseed oil. The n-9 fatty acids are primarily from plant and fungal sources. The myocardium preferentially metabolizes long chain free fatty acids such as palmitic and oleic acids or their unsaturated counterparts. Brown adipose tissue is not as selective but metabolizes free fatty acids in a way that promotes theromogenesis and they are utilized preferentially to glucose.

To the extent that free fatty acids are used in the invention, they preferably are long chain fatty acids, because they are preferentially metabolized, they are more palatable in the formulation and many can act as emulsifiers. In addition, the use of long chain fatty acids can assist in a creating a more pleasing texture.

Free fatty acids of various types can be esterfied to glycerol to form mono-, di- and triglycerides. Many of these can be obtained from a variety of vegetable and animal sources consistent with the Food and Drug Administration (FDA) requirements for food-grade products. In some circumstances, it is possible to use higher levels of food grade di- and triglycerides in the formulation so when ingested, a more prolonged peak blood concentration of lipid-containing compounds results.

These lipid materials may include fatty acids, their glycerol esters as well as carrier-bound lipids (e.g., lipoproteins). A prolonged peak blood level may more optimally reduce brown adipose tissue and myocardial FDG-uptake. It is also possible to use structured lipids to engineer optimum delivery of the lipids. For example, by using a structured lipid having a long chain unsaturated fatty acid at the 2 position and medium chain fatty acids at the 1 and 3 positions, the best delivery of the long chain fatty acid can be achieved. Similarly, if a medium chain triglyceride is used, it can be absorbed through either the portal or lymphatic pathway, yielding faster and better bioavailability.

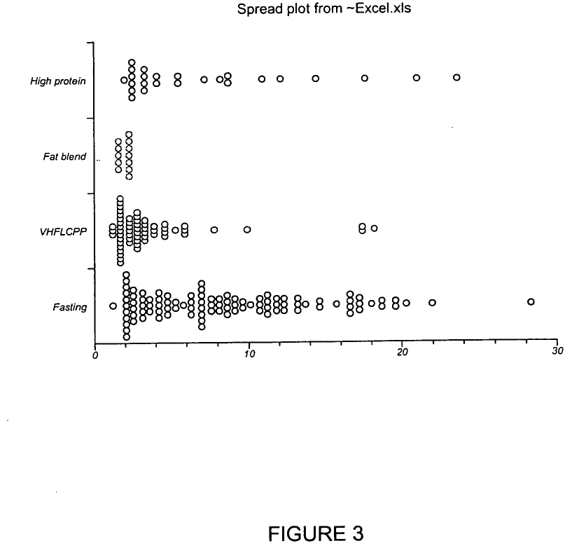

FIG. 3 is a spreadsheet showing the uptake of FDG by the myocardium after ingestion of three formulations compared with fasting after a low carbohydrate meal (the standard patient preparation). In the case of the formulations, they were taken 3-5 hour prior to FDG administration, while the fasting was about 8 hours prior to the scan. As can be seen from FIG. 3, FDG uptake in the myocardium is significantly reduced by the fat blend of the present invention.

TABLE 2

| | Mean max myocardial | Standard |
|---|---|---|
| Clinical protocol | FDG uptake | deviation |
| Fasting | 8.8 | 3.6 |
| VHFLCPP diet | 3.9 | 3.6 |
| Fat blend | 2.5 | 1.5 |
| Protein drink | 7.4 | 6.1 |

Table 2 shows the mean and standard deviation for each clinical protocol.

Since myocardial FDG uptake hinders the effectiveness of FDG scanning, this reduction is significant. The use of the lipid formulations and protocol of the invention rather than following the fasting protocol should reduce both false positive and false negative PET studies.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of reducing interferences in a FDG-PET scan of a patient comprising the steps of:
a. eliminating carbohydrate consumption by a patient for at least 8 hours prior to a FDG-PET scan; and
b. having said patient ingest a substantially carbohydrate-free composition comprising 5-75% lipid by volume and 25-90% carrier 1-10 hours prior to said FDG-PET scan; whereby ingestion of said composition causes a reduction in interferences in said scan.

2. The method of claim 1 wherein said composition is ingested 2-7 hours before said scan.

3. The method of claim 2 wherein said composition is ingested 3-5 hours before said scan.

4. The method of claim 1 wherein said composition is in the form of an emulsion.

5. The method of claim 4 wherein said emulsion comprises 20-60% lipid by volume.

6. The method of claim 5 wherein said emulsion comprises 30-50% lipid by volume.

7. The method of claim 1 wherein said lipid is in the form of free fatty acids, mono-, di- and triglycerides, structured lipids having the combination of lipids on the glycerol backbone, or a combination of any of the above.

8. The method of claim 1 wherein said FDG PET scan is being used to detect a tumor.

9. The method of claim 1 wherein said FDG PET scan is being used to detect cardiovascular abnormalities.

10. The method of claim 1 wherein said interference are related to the level of FDG uptake by brown adipose tissue.

11. A composition for reducing interferences in FDG-PET scans of a patient comprising:
a. 20-50% by volume lipid in the form of free fatty acids, mono-, di- and triglycerides, structured lipids having the combination of lipids on the glycerol backbone, or a combination of any of the above;
b. 50-80% by weight water-based carrier; and
c. 0.1-0.5% by weight aqueous or oil-based flavoring agent and wherein said composition is substantially free of carbohydrate.

12. The composition of claim 11 wherein said lipid is from vegetable oil sources.

13. The composition of claim 11 wherein said composition is in the form of an emulsion.

14. The composition of claim 13 wherein said composition further comprises emulsifiers.

15. The composition of claim 11 wherein said composition is substantially free of carbohydrates.

16. The composition of claim 11 further comprising 5-30% by weight protein.
